(19) United States
(12) Patent Application Publication
(10) Pub. No.: US 2023/0331784 A1
(43) Pub. Date: Oct. 19, 2023

(54) HCV Recombinant Antigen and Application

(71) Applicant: GUANGDONG FAPON BIOLOGICAL CO., LTD., Dongguan Guangdong (CN)

(72) Inventors: Peng CUI, Dongguan, Guangdong (CN); Zhiqiang HE, Dongguan, Guangdong (CN); Yuan MENG, Dongguan, Guangdong (CN); Zhongjie WEI, Dongguan, Guangdong (CN); Jing CEN, Dongguan, Guangdong (CN)

(21) Appl. No.: 18/026,604

(22) PCT Filed: Apr. 16, 2021
(86) PCT No.: PCT/CN2021/087908

§ 371 Date: Mar. 16, 2023

(30) Foreign Application Priority Data
Sep. 16, 2020 (CN) 202010976572.9

(51) Int. Cl. C07K 14/18; C12N 15/63; G01N 33/576

(52) U.S. Cl. CPC C07K 14/1833; C12N 15/63; G01N 33/5767; G01N 2333/186; G01N 2469/20

(57) ABSTRACT

The present disclosure relates to an HCV recombinant antigen and use of the same. Provided is an HCV recombinant antigen, which includes at least two NS3 antigens, at least one NS4 antigen, and at least one core antigen. Further provided are a nucleic acid encoding the HCV recombinant antigen, an expression vector containing the nucleic acid, a host cell containing the expression vector, a conjugate containing the HCV recombinant antigen, and a kit containing the HCV recombinant antigen, and the like.

### CROSS-REFERENCE TO RELATED APPLICATION

This application claims the benefit of the priority of the Chinese Patent Application No. 202010976572.9, filed to the China National Intellectual Property Administration on Sep. 16, 2020 and entitled “HCV Recombinant Antigen and Application”, the disclosure of which is hereby incorporated by reference in its entirety.

### TECHNICAL FIELD

The present disclosure relates to the field of Hepatitis C Virus (HCV) detection. Specifically, the present disclosure relates to an HCV recombinant antigen, which may be used for detecting the presence of an HCV antibody in the sample of a subject. The present disclosure further relates to nucleic acid encoding the HCV recombinant antigen and the mutant thereof, and a related vector, a host cell, an immunoassay method and a detection kit.

### BACKGROUND

Viral hepatitis C, referred to as hepatitis C, is a disease caused by infection with the HCV (hepatitis C virus) and is mainly transmitted by blood or body fluids. The World Health Organization estimates that approximately 180 million people worldwide are infected with the hepatitis C, and the global HCV infection rate is about 3%. The prevalence of anti-HCV positivity in our healthy population is 0.7%-3.1%, about 38 million people. Due to various factors such as the biological characteristics of the virus and host immune functions, it is often difficult for the immunity of a body to effectively clear the virus, leading to the development of chronic hepatitis in about 80% of HCV-infected patients, of which 10%-20% will develop into cirrhosis, and 1%-5% of patients with cirrhosis will develop into hepatocellular carcinoma each year. As a result, the prevalence of chronic HCV infection has become a significant socioeconomic burden in countries around the world.

The HCV is a single-stranded positive-stranded RNA virus. The whole genome is about 9.5 kb long and may be divided into 3 regions, which are a 5′Untranslated Region (5′UTR), an Open Reading Frame (ORF) and a 3′UTR, and the order of the above regions is 5′UTR-C-E1-E2-p7-NS2-NS3-NS4-NS5-3′UTR. The 5′UTR is a highly conserved region, which is an initiation site of viral translation and plays a very important role in an HCV replication process. The ORF includes structural protein regions C, E1 and E2, and non-structural protein regions P7 and N52-NS5. Envelope regions (E1 and E2) and a core region (C) encode virus particles, and the non-structural protein regions play an important role in virus replication and virus protein synthesis. The core region, and NS2, NS3, NS4A, NS5A and NS5B of a non-structural protein part are important target points of immunodiagnosis research at present.

The gene encoding an HCV core protein is located at nucleotide loci 342-914 in the HCV genome and encodes 191 amino acids, and the amino acid sequence is very conservative. The HCV core protein is a viral protein having various biological functions; and in addition to having a function of assembling the virus particles, the protein plays an important role in HCV proliferation and pathogenesis, and is an important marker for reflecting HCV infection. The HCV core protein is produced before the positive conversion of antibodies in the body after HCV infection, and is an important indicator of HCV replication and HCV viral load in patients. Since the protein is highly conserved and highly immunogenic, the protein is widely used in HCV diagnostics and vaccine studies. The NS3 gene is located at nucleotide loci 3300-5200 of the HCV whole genome sequence and encodes 630 amino acids. The NS3 protein has a protease function, 189 amino acids at its N terminal have serine protease activity, and 442 amino acids at a C terminal have the activity of nucleoside triphosphatase (NTPase) and RNA helicase. During HCV infection, anti-NS3 antibodies are the first to appear. The NS3 protein is highly antigenic, and almost all HCV-infected patients produce high-titer and specific anti-NS3 antibodies. The NS4 gene is located at nucleotides 4974-5133 of the whole HCV genome and encodes two proteins NS4A and NS4B. NS4A is a short peptide of 54 amino acids; and NS4B is a 27 kDa-sized transmembrane protein localized to an ER membrane and has strong hydrophobicity. The NS4 region includes at least two immunodominant sequence sites and is highly antigenic, and the vast majority of HCV-infected patients produce antibodies against the region. The NS5 gene coding region is 3117 nucleotides long, encodes two proteins NS5A and NA5B, and is the longest coding region in the HCV genome.

With the development of a genetic engineering technology, the molecular biology of HCV viral genes has become increasingly mature and been applied to diagnostic reagents. An HCV diagnostic reagent mainly uses gene fragments, such as core, NS3 and NS4, on different detection platforms. In the diagnostic reagent, HCV diagnostic raw materials are mostly in the mode of individual HCV-core, individual NS3, chimeric core+NS3, chimeric NS3+core, and chimeric NS3+NS4. Due to the diversity of clinical samples and the different timing and amount of antibodies produced against different sections, a single fragment or the chimerism of only two fragments easily leads to missed detection of clinical tests.

Therefore, a product that can avoid missed detection of clinical tests and detect HCV with high sensitivity and high specificity is still needed in the art.

### SUMMARY

The present disclosure provides a Hepatitis C Virus (HCV) recombinant antigen. The HCV recombinant antigen includes at least two NS3 antigens, at least one NS4 antigen, and at least one core antigen. For example, the HCV recombinant antigen may include at least two NS3 antigens, at least two NS4 antigens, and at least one core antigen. For example, the HCV recombinant antigen may include at least two NS3 antigens, at least three NS4 antigen, and at least one core antigen. For example, the number of the NS3 antigens in the HCV recombinant antigen may be two, three or more, for example, the two, three or more NS3 antigens may be the same or different, preferably different. For example, the number of the NS4 antigens in the HCV recombinant antigen is one, two, three or more, for example, preferably the two, three or more NS4 antigens are different. For example, the number of the core antigens in the HCV recombinant antigen may be one, two, three or more, for example, preferably the two, three or more core antigens may be the same or different, preferably different.

In one or more implementations, the sensitivity of the HCV recombinant antigen for detecting an HCV antibody is not less than 94.0%, for example, not less than 97.0%, not less than 98.0%, and not less than 99.0%.

In one or more implementations,
(1) the NS3 antigen includes polypeptide selected from positions 1075-1657 of an HCV amino acid sequence, for example, the length of the polypeptide is 41-583 amino acids, for example, 50, 60, 70, 80, 90, 100, 150, 200, 250, 300, 350, 400, 450, 500 or 550 amino acids; for example, the NS3 antigen includes polypeptide of which initiation site is at positions 1075-1380 of the HCV amino acid sequence (for example, the initiation site is at position 1075, 1192, 1201, 1230, 1377, 1380 or any position of the positions 1075-1380) and a termination site at positions 1420-1657 of the HCV amino acid sequence (for example, the termination site is at position 1420, 1443, 1451, 1465, 1478, 1657 or any position of the positions 1420-1657); for example, the NS3 antigen includes positions 1380-1420, 1075-1657, 1230-1465, 1377-1443, 1192-1451, 1192-1478, 1377-1443, 1201-1465, 1192-1608 or 1192-1517 of the HCV amino acid sequence; for example, the NS3 antigen includes a sequence selected from SEQ ID NO:1-14; for example, the NS3 antigen includes a sequence selected from sequences having at least 95%, at least 96%, at least 97%, at least 98%, or at least 99% identity to SEQ ID NO:1-14;
(2) the NS4 antigen includes polypeptide selected from positions 1658-1949 of an HCV amino acid sequence, for example, the length of the polypeptide is 38-292 amino acids, for example, 40, 50, 60, 70, 80, 90, 100, 150, 200 or 250 amino acids; for example, the NS4 antigen includes polypeptide of which initiation site is at positions 1658-1698 of the HCV amino acid sequence (for example, the initiation site is at position 1658, 1691, 1693, 1695, 1698 or any position of the positions 1658-1698) and a termination site at positions 1735-1949 of the HCV amino acid sequence (for example, the termination site is at position 1735, 1740, 1741, 1749, 1799, 1931, 1935, 1949 or any position of the positions 1735-1949); for example, the NS4 antigen includes positions 1698-1735, 1658-1949, 1691-1749, 1695-1741, 1693-1740, 1698-1931, 1691-1799 or 1929-1935 of the HCV amino acid sequence; for example, the NS4 antigen includes a sequence selected from SEQ ID NO:15-22; for example, the NS4 antigen includes a sequence selected from sequences having at least 95%, at least 96%, at least 97%, at least 98%, or at least 99% identity to SEQ ID NO:15-22; and/or
(3) the core antigen includes polypeptide selected from positions 1-179 of an HCV amino acid sequence, for example, the length of the polypeptide is 27-179 amino acids, for example, 30, 40, 50, 60, 70, 80, 90, 100 or 150 amino acids; for example, the core antigen includes polypeptide of which initiation site is at positions 1-8 of the HCV amino acid sequence (for example, the initiation site is at position 1, 8 or any position of the positions 1-8) and a termination site at positions 34-179 of the HCV amino acid sequence (for example, the termination site is at position 34, 48, 53, 60, 80, 130, 179 or any position of the positions 34-179); for example, the core antigen includes positions 1-34, 8-34, 1-179, 1-130, 1-80, 1-53, 1-48 or 8-60 of the HCV amino acid sequence; for example, the core antigen includes a sequence selected from SEQ ID NO:24-34; for example, the core antigen includes a sequence selected from sequences having at least 95%, at least 96%, at least 97%, at least 98%, or at least 99% identity to SEQ ID NO:24-34.

In one or more implementations, at least any one of cysteine in the NS3 antigen is mutated into another amino acid, for example, mutated into G, A or S. For example, at least any one (at least one, two, three, four, five or six) of cysteine of the NS3 antigen in position 1305/1315/1318/1394/1400/1454 of the corresponding HCV amino acid sequence is mutated into another amino acid, for example, mutated into G, A or S.

In one or more implementations, the specificity of the HCV recombinant antigen for detecting an HCV antibody is not less than 98.0%.

In one or more implementations, the sensitivity of the HCV recombinant antigen for detecting the HCV antibody is not less than 98.0%, for example, not less than 99.0%.

In one or more implementations, the specificity and sensitivity of the HCV recombinant antigen for detecting the HCV antibody are both not less than 98.0%, for example, not less than 99.0%.

In one or more implementations, the NS3 antigen, the NS4 antigen and the core antigen are directly connected or connected optionally by means of one or more linkers. For example, the linker is (G)n, (GS)n, (SG)n, (GGGS)n or (GGGGS)n, wherein n is 1, 2, 3, 4, 5, 6, 7, 8, 9, 10 or a larger integer.

In one or more implementations, the HCV recombinant antigen includes N- and/or C-terminal modifications, for example, histidine tags, biotinylation and/or detectable marker modifications.

In one or more implementations, the HCV includes an HCV genotype 1a, 1 b, 2a, 2b, 2c, 2d, 3a, 3b, 3c, 3d, 3e, 3f, 4a, 4b, 4c, 4d, 4e, 4f, 4g, 4h, 4i, 4j, 5a or 6a.

In one or more implementations, antigens contained in the HCV recombinant antigen described here are fused in the order of the NS3 antigen, the NS4 antigen, and the core antigen from an N-terminal to a C-terminal. For example, when the HCV recombinant antigen includes two NS3 antigens, two NS4 antigen and one core antigen, the included antigens may be fused in the order of a first NS3 antigen, a second NS3 antigen, a first NS4 antigen, a second NS4 antigen and the core antigen, and so on when the HCV recombinant antigen includes a combination of other antigens.

The present disclosure provides a nucleic acid encoding the HCV recombinant antigen described here.

The present disclosure provides an expression vector containing the nucleic acid described here.

The present disclosure provides a host cell containing the expression vector described here, for example, an Escherichia coli cell.

The present disclosure provides a conjugate, which includes the HCV recombinant antigen described here.

In one or more implementations, the conjugate described here further includes a solid support, a detectable marker or a binding partner which is conjugated with the HCV recombinant antigen. For example, the HCV recombinant antigen in the conjugate may be directly or indirectly conjugated. For example, the solid support includes magnetic particles, a microtiter plate or a cellulose membrane. For example, the detectable marker may be a metal particle, a fluorescent marker, a chromophore marker, an electron-dense marker, a chemiluminescent marker, a radioactive marker, or an enzyme marker; and for example, the detectable marker may be colloidal gold, radio isotope, fluorophores, a spin marker, or a bacteriophage marker; for example, the detectable marker may be rhodamine, fluorescein, acridinium ester, luciferase, horseradish peroxidase, alkaline phosphatase, β-galactosidase, glucoamylase, lysozyme, carbohydrate oxidase, glucose oxidase, galactose oxidase or a glucose-6-phosphate dehydrogenase marker. For example, the binding partner includes biotin, streptavidin or avidin.

The present disclosure further provides a kit, which contains the HCV recombinant antigen described here or the conjugate described here. In some implementations, the kit may contain another HCV antigen, and the HCV antigen includes epitope that is immunoreactive with an HCV antibody. In some implementations, the HCV recombinant antigen described in the present disclosure and another HCV antigen may be co-encapsulated on the same solid phase or separately encapsulated on separate solid phases.

In one or more implementations, the kit described here further contains a detection antibody. In some implementations, the detection antibody may be an antibody that detects human antibodies. In some implementations, the kit may include an anti-HCV antibody. In some implementations, the detection antibody and/or the anti-HCV antibody may be marked with a detectable marker.

The present disclosure further provides use of the HCV recombinant antigen described here or the conjugate described here in preparation of a kit for detecting an HCV antibody or antigen from a sample of a subject.

In one or more implementations, the sample includes biological tissue, cells or body fluids in a healthy or pathological state, such as blood samples, for example, plasma, serum or blood products, such as semen or vaginal secretions.

The present disclosure further provides use of the HCV recombinant antigen described here or the conjugate described here for detecting an HCV antibody or antigen from a sample of a subject.

The present disclosure further provides a method for detection an HCV antibody or antigen in a sample of a subject. The method includes contacting the HCV recombinant antigen described here or the conjugate described here with a sample of a subject.

The present disclosure further provides a method for diagnosing hepatitis C. The method includes the following operations.

The HCV recombinant antigen described here or the conjugate described here is used to detect an HCV antibody or antigen from a sample of a subject.

A detection result is analyzed.

### DETAILED DESCRIPTION OF THE EMBODIMENTS

In order to make the purposes, technical solutions and advantages of the implementations of the present disclosure clearer, the technical solutions in the implementations of the present disclosure will be clearly and completely described below. If specific conditions are not indicated in the implementations, the implementations are carried out in accordance with the conventional conditions or the conditions recommended by manufacturers. Reagents or instruments used are conventional products that may be purchased commercially if the manufacturers are not specified.

In some implementations, the inventor delved into the construction of a brand-new HCV genetically engineered recombinant protein by means of combining or chimerizing different fragments during the process of cloning and constructing an HCV target chimeric protein, effectively solving the problem of insufficient sensitivity of clinical HCV antibody detection.

In some implementations, in the present disclosure, in order to solve the problem of relatively low sensitivity of clinical HCV antibody detection, a novel combined HCV genetically engineered recombinant antigen is constructed after extensive exploration. By means of the HCV recombinant antigen of the present disclosure, the sensitivity of detecting core, NS3, and NS4 antibodies is effectively improved, thereby avoiding the problem of clinical missed detection.

In some implementations, in the present disclosure, in order to further improve the specificity of clinical HCV antibody detection, partial sites of NS3 sub-fragments in the HCV recombinant antigen are mutated, such that the specificity of clinical HCV antibody detection is effectively improved, thereby reducing the occurrence of false positives.

The recombinant HCV antigen of the present disclosure may be prepared by means of any appropriate method known in the art. For example, in some implementations, a nucleic acid encoding the recombinant HCV antigen of the present disclosure may be prepared, and cloned to any appropriate vector such as plasmid, phage and cosmid; and then recombinant molecules are expressed by means of an appropriate expression system or a host (for example, insect, mammalian, bacterial, viral, and yeast expression systems). In some implementations, host cells suitable for recombinant expression are well known in the art, including, but not limited to: animal cells, such as Chinese Hamster Ovary (CHO) cells, HeLa cells and human embryonic kidney cells; bacterial host cells, such as Escherichia coli, Bacillus subtilis and streptococcus cells; and yeast host cells, such as brewer's yeast. In some implementations, the recombinant HCV antigen may be prepared by using one or more linkers to connect antigen fragments. The linker used here includes a natural or artificial polypeptide sequence that links target polypeptide sequences. The linker may have a length of about 4-50 amino acids, for example, a length of about 6-30 amino acids.

In some implementations, the genotype of HCV in the present disclosure is not specifically limited, and may include, for example, HCV genotype 1a, 1 b, 2a, 2b, 2c, 2d, 3a, 3b, 3c, 3d, 3e, 3f, 4a, 4b, 4c, 4d, 4e, 4f, 4g, 4h, 4i, 4j, 5a or 6a. In some implementations, the recombinant HCV antigen of the present disclosure may include further modifications, for example, label modifications. The label modifications of the recombinant HCV antigen of the present disclosure is not particularly limited, for example, may be a protein purification label, for example, an affinity label, such as a biotin label. As is well known in the art, separation of target HCV antibodies may be achieved by specifically binding the target HCV antibodies and labeled recombinant HCV antigens and performing separation by identifying a labeled binding partner.

In some implementations, the HCV antibody in the sample of the subject identifies epitope in the recombinant HCV antigen of the present disclosure, such that the recombinant HCV antigen of the present disclosure may be used in immunoassays to detect the HCV antibody in the sample of the subject. In some implementations, the recombinant HCV antigen of the present disclosure may be used for immunoassays, such as ELISA, fluorescence immunochromatography, colloidal gold immunochromatography, chemiluminescence assay, electrochemiluminescence assay, Indirect Immunofluorescence Assay (IFA), Radioimmunoassay (RIA) and other non-enzyme-linked antibody binding tests or methods. In some implementations, the recombinant HCV antigen of the present disclosure may be used as a capture antigen or a detection antigen, or is used as the detection antigen and the capture antigen at the same time. In some implementations, another antigen paired with the recombinant HCV antigen of the present disclosure may be the same or different, as long as each fragment in the HCV recombinant antigen of the present disclosure is included. For example, in some implementations, another antigen paired with the recombinant HCV antigen of the present disclosure may be the identical antigen as the recombinant HCV antigen of the present disclosure, or may be a different antigen including corresponding fragments of the recombinant HCV antigen of the present disclosure. In some implementations, for example, in an ELISA solution, the recombinant HCV antigen may be used as the capture antigen to encapsulate a solid phase such as magnetic beads, so as to capture the HCV antibody in the sample, and then a result is read after color development. In some implementations, antigens or antibodies used in immunoassays may be fixed on a surface, for example, on a solid support, such as plastic, a membrane such as a nitrocellulose membrane, glass, a magnetic bead or a metal support. In some implementations, the sample of the subject is in contact with the solid support, and then is in contact with the detection antibody or the detection antigen with a detectable marker for color development after identification. Herein, the sample of the subject may include biological tissue, cells or body fluids in a healthy or pathological state, such as blood samples, for example, plasma, serum or blood products, such as semen or vaginal secretions.

In some implementations, the detection antigen or the detection antibody (for example, an anti-human IgG antibody or an anti-human IgM antibody) may be marked with the detectable marker. In some implementations, the detectable marker used for marking the antigens or the antibodies is not particularly limited. In some implementations, the marker may include, but not limited to, a fluorescent marker, a chromophore marker, an electron-dense marker, a chemiluminescent marker, a radioactive marker, and an indirect marker such as enzyme or ligand, for example, indirect detection is achieved by means of enzymatic reaction or molecular interaction. In some implementations, exemplary markers include, but are not limited to, radioisotopes, fluorophores, rhodamine and derivatives thereof, luciferase, fluorescein, Horseradish Peroxidase (HRP), alkaline phosphatase, β-galactosidase, glucoamylase, lysozyme, carbohydrate oxidase such as glucose oxidase, galactose oxidase and glucose-6-phosphate dehydrogenase, biotin/avidin, spin markers, bacteriophage markers, and the like.

In some implementations, the present disclosure provides a method, for example, immunoassay for detecting the presence of an anti-HCV antibody in a sample of a subject. The method may include: contacting the recombinant HCV antigen of the present disclosure with the sample; insofar as there is the HCV antibody in the sample, forming a complex of the HCV antibody and the recombinant HCV antigen; and detecting the presence of the complex, wherein the presence of the complex indicates that there is the HCV antibody in the sample. In some implementations, the complex may be detected by determining the detectable marker (for example, the fluorescent marker). In some implementations, the sample containing or suspected to contain the HCV antibody may be in contact with the recombinant HCV antigen of the present disclosure and at least one detection antibody (for example, a second detection antibody or a third detection antibody, for example, an anti-IgG antibody or an anti-IgM antibody with the detectable marker) at the same time or in any order. In some implementations, the method and/or kit of the present disclosure is applicable to any appropriate automatic or semi-automatic system.

In some implementations, the present disclosure provides a kit containing the recombinant HCV antigen of the present disclosure, for example, a kit used for detecting the presence of an HCV antibody in a sample of a subject. In some implementations, the kit includes a reagent suitable for performing immunoassay. In some implementations, the kit may include a specification for the use of an immunodiagnostic reagent (for example, a conjugate containing a recombinant HCV antigen) of the present disclosure in the immunoassay for detecting the HCV antibody. In some implementations, the kit may include a calibrator or a control, for example, a standard or control HCV antibody. In some implementations, the recombinant HCV antigen or conjugate of the present disclosure is contained on a container, such as a test tube, a microplate or a test strip, in the kit. In some implementations, the kit may further include a solid support, such as a magnetic bead, a test tube, a microplate, a cuvette, a thin film, a filter paper, an injection syringe, a pipette, a buffer solution such as a determination buffer solution, a washing buffer solution, a pretreatment reagent, a substrate solution with the detectable marker such as an enzyme marker.

In some implementations, the present disclosure includes a test strip containing the recombinant HCV antigen, for example, a lateral chromatography test strip. In some implementations, the test strip contains the recombinant HCV antigen encapsulated on the solid phase, at least one detection antibody with the detectable marker (such as colloidal gold) or at least one detection antigen. In some implementations, the test strip contains the recombinant HCV antigen with the detectable marker, at least one detection antibody encapsulated on the solid phase or at least one detection antigen. In some implementations, by means of the present disclosure, the HCV antibody in the subject may be rapidly and accurately detected by means of visual observation or a fully-automated chemiluminescent instrument. In some implementations, the kit is based on double antigen sandwich immunoassay. For example, in some implementations, the antibody in the sample is captured by the recombinant HCV antigen encapsulated on the solid phase; or the antibody in the sample is detected by the recombinant HCV antigen which is marked by the detectable marker. In some implementations, the kit is based on indirect immunoassay. For example, in some implementations, the antibody in the sample is captured by the recombinant HCV antigen encapsulated on the solid phase. In some implementations, an excitation solution is added; a luminous value is measured by the fully-automated chemiluminescent instrument; the luminous value is positively correlated with the total concentration of the antibody in the sample; and negative and positive are determined by comparing the luminous value with a critical value. The term antibody used here, such as the detection antibody, is not particularly limited, may include, for example, a monoclonal antibody, a polyclonal antibody, a multi-specific antibody, a human antibody, a humanized antibody, a recombinant antibody, a chimeric antibody, a single-chain antibody and a single-domain antibody, or may include functional fragments of the antibody such as a Fab fragment, a F(ab′) fragment, a Fab′-SH fragment, a F(ab′)2 fragment, a Fd fragment, a Fv fragment, a single-chain Fv fragment (scFv), a dAb fragment and an isolated Complementary Decision Region (CDR), an anti-idiotypic antibody, a bifunctional antibody, a double-domain antibody, or the like.

In some implementations, the method and/or kit described here may be used for detecting the presence of the HCV antibody in the sample of the subject, measuring the volume or concentration of the HCV antibody, monitoring progression of diseases, monitoring a treatment effect, and/or determining the onset of hepatitis in subjects or onset risks.

In one or more implementations, the sensitivity of the HCV recombinant antigen for detecting an HCV antibody is not less than 94.0%, for example, not less than 95.0%, not less than 96.0%, not less than 97.0%, not less than 98.0% or not less than 99.0%.

In one or more implementations, the specificity of the HCV recombinant antigen for detecting an HCV antibody is not less than 98.0%, for example, not less than 98.1%, not less than 98.2%, not less than 98.3%, not less than 98.4%, not less than 98.5%, not less than 98.6%, not less than 98.7%, not less than 98.8%, not less than 98.9%, not less than 99.0%, not less than 99.1%, not less than 99.2%, not less than 99.3%, not less than 99.4%, not less than 99.5%, not less than 99.6%, not less than 99.7%, not less than 99.8% or not less than 99.9%.

In one or more implementations, the sensitivity of the HCV recombinant antigen for detecting the HCV antibody is not less than 98.0%, for example, not less than 99.0%.

In one or more implementations, the specificity and sensitivity of the HCV recombinant antigen for detecting the HCV antibody are both not less than 98.0%, for example, not less than 99.0%.

The present disclosure further provides use of the HCV recombinant antigen described here or the conjugate described here for detecting an HCV antibody or antigen from a sample of a subject.

The present disclosure further provides a method for detection an HCV antibody or antigen in a sample of a subject. The method includes contacting the HCV recombinant antigen described here or the conjugate described here with a sample of a subject.

The present disclosure further provides a method for diagnosing hepatitis C. The method includes the following operations.

The HCV recombinant antigen described here or the conjugate described here is used to detect an HCV antibody or antigen from a sample of a subject.

A detection result is analyzed.

The method and/or products in the present disclosure solve one or more problems of low sensitivity and low specificity. Therefore, compared with existing methods, the method of the present disclosure has the following one or more advantages: sensitivity is improved, and/or specificity is improved.

The term fusion protein used here refers to a protein or polypeptide that is expressed in fusion with the HCV antigen. The protein or the polypeptide may be used for affinity or marking (directly or indirectly). For example, in some implementations, the fusion protein is expressed in fusion with the HCV antigen, and the marking of the marker (such as colloidal gold) is achieved by means of specific binding between the anti-fusion protein antibody and the fusion protein.

### Embodiment 1 Construction of a Vector Containing a Fusion Protein Encoding Sequence

In this embodiment, a fusion protein is selected as a specific protein for clone construction, and a sequence of the fusion protein is shown as follows; a coding sequence of the fusion protein may be obtained by means of E. coli, PET32A and gene synthesis; and at the same time, codon optimization may be performed to reach an optimal expression state. In this embodiment, the following fusion protein sequence is used to construct an expression vector containing an HIS tag, introduce (GGGGS)₃ at the 3′ end of the fusion protein sequence, and reserve a BgIII/EcoRI endonuclease site.

An amino acid sequence of the fusion protein:

(SEQ ID NO: 35)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLA

### Embodiment 2 Construction of Recombinant Protein Expression Plasmids for HCV Genetic Engineering

Gene segments of an HCV recombinant protein are designed; and the gene fragments may be spliced by means of restriction endonuclease and a T4 DNA ligase tool enzyme in a manner of enzyme digestion and connection, or may be spliced in the form of primer bridging by means of bridge PCR. Sections may be connected by means of or without linkers, and the linkers may use the form of common linkers, for example, GGGGSGGGGSGGGGS(SEQ ID NO:36). The above gene fragments are constructed into the expression vector of Embodiment 1 by means of the restriction endonuclease and the T4 DNA ligase in the manner of enzyme digestion and connection.

Specifically, the following HCV recombinant antigens are prepared. Unless otherwise stated, each recombinant HCV antigen is prepared by fusing each described antigen region in an order from an N-terminal to a C-terminal. The antigen regions are connected by means of or without linkers (GGGGSGGGGSGGGGS) (see specific description for each HCV recombinant antigen). Sequences of the antigen regions are described in detail below.

HCV-1 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-1, NS3-1, NS4-1, NS4-2, NS4-3, and Core-1, without linkers.

HCV-1B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-1, NS3-1, NS4-1, NS4-2, NS4-3B, and Core-1, without linkers.

HCV-2 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-2, NS3-2, NS4-2, NS4-3, NS4-4, and Core-2, without linkers.

HCV-2B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-2B, NS3-2B, NS4-2, NS4-3B, NS4-4, and Core-2B, without linkers.

HCV-3 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-1, NS3-2, NS4-1, NS4-2, NS4-5, and Core-3, without linkers.

HCV-3B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-1, NS3-2B, NS4-1, NS4-2, NS4-5, and Core-3B, without linkers.

HCV-4 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-5, NS3-4, NS4-6, NS4-5, NS4-4, and Core-4, without linkers.

HCV-4B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-5B, NS3-4B, NS4-6, NS4-5, NS4-4, and Core-4B, without linkers.

HCV-5 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-3, NS3-7, NS4-3, NS4-4, NS4-5, and Core-6, without linkers.

HCV-5B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-3B, NS3-7B, NS4-3B, NS4-4, NS4-5, and Core-6, without linkers.

HCV-6 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-1, NS3-7, NS4-7, NS4-1, NS4-4, and Core-5, without linkers.

HCV-6B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-1, NS3-7B, NS4-7, NS4-1, NS4-4, and Core-5, without linkers.

HCV-7 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-2, NS3-6, NS4-2, NS4-7, NS4-6, and Core-6, without linkers.

HCV-7B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-2B, NS3-6B, NS4-2, NS4-7, NS4-6, and Core-6, without linkers.

HCV-8 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-4, NS3-5, NS4-6, NS4-3, NS4-1, and Core-7, without linkers.

HCV-8B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-4B, NS3-5B, NS4-6, NS4-3B, NS4-1, and Core-7, without linkers.

HCV-9 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-6, NS3-3, NS4-4, NS4-2, NS4-5, and Core-8, without linkers.

HCV-9B recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-6B, NS3-3B, NS4-4, NS4-2, NS4-5, and Core-8, without linkers.

HCV-5C recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-3, NS3-7, NS4-3, NS4-4, NS4-5, and Core-6, with linkers.

HCV-4C recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-5, NS3-4, NS4-6, NS4-5, NS4-4, and Core-4, with linkers.

HCV-10 recombinant antigen is prepared according to the above description, and is a fusion polypeptide fusing a first NS3 region, a second NS3 region, a first NS4 region, a second NS4 region, a third NS4 region and a CORE region from the N-terminal to the C-terminal, and the regions are respectively in an order: NS3-4, NS3-6, NS4-2, NS4-5, NS4-3, and Core-7, with linkers.

HCV-11 recombinant antigen is prepared according to the above description, and is a fusion polypeptide fusing a first NS3 region, a second NS3 region and a CORE region from the N-terminal to the C-terminal, and the regions are respectively in an order: NS3-5, NS3-4, and Core-4, without linkers.

HCV-12 recombinant antigen is prepared according to the above description, and is a fusion polypeptide fusing an NS3 region and a CORE region from the N-terminal to the C-terminal, and the regions are respectively in an order: NS3-5 and Core-4, without linkers.

HCV-13 recombinant antigen is prepared according to the above description, and is a fusion polypeptide fusing a first NS3 region, a second NS3 region, an NS4 region and a CORE region from the N-terminal to the C-terminal, and the regions are respectively in an order: NS3-5, NS3-4, NS4-5 and Core-4, without linkers.

HCV-14 recombinant antigen is prepared according to the above description, and is a fusion polypeptide fusing a first NS3 region, a second NS3 region, a first NS4 region, a second NS4 region and a CORE region from the N-terminal to the C-terminal, and the regions are respectively in an order: NS3-5, NS3-4, NS4-5, NS4-4, and Core-4, without linkers.

HCV-15 recombinant antigen is prepared according to the above description, and is a fusion polypeptide fusing a first NS3 region and a second NS3 region from the N-terminal to the C-terminal, and the regions are respectively in an order: NS3-5 and NS3-4, without linkers.

HCV-16 recombinant antigen is prepared according to the above description, and is a fusion polypeptide fusing an NS3 region, a CORE region and an NS4 region from the N-terminal to the C-terminal, and the regions are respectively in an order: NS3-8, Core-8 and NS4-8, without linkers.

HCV-17 recombinant antigen is prepared according to the above description, and is a fusion polypeptide fusing an NS3 region, a first NS4 region, a second NS4 region, a third NS4 region and a CORE region from the N-terminal to the C-terminal, and the regions are respectively in an order: NS3-1, NS4-1, NS4-2, NS4-3 and Core-1, without linkers.

HCV-18 recombinant antigen is prepared according to the above description, and is a fusion polypeptide fusing an NS3 region, a first NS4 region, a second NS4 region and a CORE region from the N-terminal to the C-terminal, and the regions are respectively in an order: NS3-1, NS4-1, NS4-2 and Core-1, without linkers.

HCV-19 recombinant antigen is prepared according to the above description, and is a fusion polypeptide fusing an NS3 region, a first NS4 region, a second NS4 region, a third NS4 region and a CORE region from the N-terminal to the C-terminal, and the regions are respectively in an order: NS3-2, NS4-2, NS4-3, NS4-4 and Core-2, without linkers.

HCV-20 recombinant antigen is prepared according to the above description, and is a fusion polypeptide fusing an NS3 region, a first NS4 region, a second NS4 region and a CORE region from the N-terminal to the C-terminal, and the regions are respectively in an order: NS3-2, NS4-2, NS4-3 and Core-2, without linkers.

Sequence Information:

NS3-1 (which is located in positions 1380-1420 of the HCV amino acid sequence, SEQ ID
NO: 1):
IPIEAIRGGRHLIFCHSKKKCDELAAKLSSLGLNAVAYYRG
NS3-2 (which is located in positions 1075-1657 of the HCV amino acid sequence, SEQ ID
NO: 2):
NGVCWTVYHGAGSKTLAGPKGPITQMYTNVDQDLVGWHRPPGARSLTPCTCGSSDLYLVTR
HADVIPVRRRGDSRGSLLSPRPVSYLKGSSGGPLLCPFGHVAGIFRAAVCTRGVAKAVDFIPVETM
ETTMRSPVFTDNSSPPAVPQTFQVAHLHAPTGSGKSTKVPAAYAAQGYKVLVLNPSVAATLGFGAY
MSKAHGVDPNIRTGVRTITTGAPITYSTYGKFLADGGCSGGAYDIIICDECHSTDSTTILGIGTVLDQ
AETAGARLVVLATATPPGSVTVPHPNIEEVALSNTGEIPFYGKAIPIEAIRGGRHLIFCHSKKKCDELA
AKLSSLGLNAVAYYRGLDVSVIPSSGDVVVVATDALMTGFTGDFDSVIDCNTCVTQTVDFSLDPTFT
IETTTVPQDAVSRSQRRGRTGRGREGIYRFVTPGERPSGMFDSSVLCECYDAGCAWYELTPAETT
VRLRAYLNTPGLPVCQDHLEFWEGVFTGLTHIDAHFLSQTKQAGDNFPYLVAYQATVCAKAQAPP
PSWDQMWKCLTRLKPTLQGPTPLLYRLGAVQNEVTLTHPITKYIMTCMSADLEVVT
NS3-2B (which is located in positions 1075-1657 of the HCV amino acid sequence, SEQ ID
NO: 3):
NGVCWTVYHGAGSKTLAGPKGPITQMYTNVDQDLVGWQAPPGARSLTPCTCGSSDLYLVTR
HADVIPVRRRGDTRGSLLSPRPVSYLKGSSGGPLLCPSGHAVGIFRAAVCTRGVAKAVDFVPVES
METTMRSPVFTDNSSPPAVPQTFQVAHLHAPTGSGKSTKVPAAYAAQGYKVLVLNPSVAATLGFG
AYMSKAHGVDPNIRTGVRTITTGGPITYSTYGKFLADGGCSGGAYDIIICDECHSTDSTTILGIGTVL
DQAETAGARLVVLATATPPGSVTVPHPNIEEVALSNTGEIPFYGKAIPIETIKGGRHLIFCHSKKKCDE
LAAKLSSLGLNAVAYYRGLDVSVIPTSGDVVVVATDALMTGFTGDFDSVIDCNTCVTQTVDFSLDPT
FTIETTTVPQDAVSRSQRRGRTGRGRGGIYRFVTPGERPSGMFDSSVLCECYDAGCAWYELTPA
ETSVRLRAYLNTPGLPVCQDHLEFWESVFTGLTHIDAHFLSQTKQAGDNFPYLVAYQATVCARAQA
PPPSWDQMWKCLTRLKPTLHGPTPLLYRLGAVQNEVTLTHPITKYIMACMSADLEVVT
NS3-3 (which is located in positions 1230-1465 of the HCV amino acid sequence, SEQ ID
NO: 4):
APTGSGKSTKVPAAYAAQGYKVLVLNPSVAATLGFGAYMSKAHGVDPNIRTGVRTITTGAPITY
STYGKFLADGGCSGGAYDIICDECHSTDSTTILGIGTVLDQAETAGARLVVLATATPPGSVTVPHPNI
EEVALSNTGEIPFYGKAIPIEAIRGGRHLIFCHSKKKCDELAAKLSSLGLNAVAYYRGLDVSVIPSSG
DVVVVATDALMTGFTGDFDSVIDCNTCVTQTVDFS
NS3-3B (which is located in positions 1230-1465 of the HCV amino acid sequence, SEQ ID
NO: 5):
APTGSGKSTKVPAAYAAQGYKVLVLNPSVAATLGFGAYMSKAHGVDPNIRTGVRTITTGSPITY
STYGKFLADGGCSGGAYDIIICDECHSTDATSILGIGTVLDQAETAGARLVVLATATPPGSVTVSHPN
IEEVALSTTGEIPFYGKAIPLEVIKGGRHLIFCHSKKKCDELAAKLVALGINAVAYYRGLDVSVIPTSGD
VVVVSTDALMTGFTGDFDSVIDCNTCVTQTVDFS
NS3-4 (which is located in positions 1377-1443 of the HCV amino acid sequence, SEQ ID
NO: 6):
GKAIPIEAIRGGRHLIFCHSKKKCDELAAKLSSLGLNAVAYYRGLDVSVIPSSGDVVVVATDALM
TG
NS3-4B (which is located in positions 1377-1443 of the HCV amino acid sequence, SEQ ID
NO: 7):
GKAIPLEVIKGGRHLIFCHSKKKCDELAAKLVALGINAVAYYRGLDVSVIPTSGDVVVVSTDALM
TG
NS3-5 (which is located in positions 1192-1451 of the HCV amino acid sequence, SEQ ID
NO: 8):
AVDFIPVETMETTMRSPVFTDNSSPPAVPQTFQVAHLHAPTGSGKSTKVPAAYAAQGYKVLVL
NPSVAATLGFGAYMSKAHGVDPNIRTGVRTITTGAPITYSTYGKFLADGGCSGGAYDIIICDECHST
DSTTILGIGTVLDQAETAGARLVVLATATPPGSVTVPHPNIEEVALSNTGEIPFYGKAIPIEAIRGGRH
LIFCHSKKKCDELAAKLSSLGLNAVAYYRGLDVSVIPSSGDVVVVATDALMTGFTGDFDSV
NS3-5B (which is located in positions 1192-1451 of the HCV amino acid sequence, SEQ ID
NO: 9):
AVDFIPVENLETTMRSPVFTDNSSPPAVPQSFQVAHLHAPTGSGKSTKVPAAYAAQGYKVLVL
NPSVAATLGFGAYMSKAHGVDPNIRTGVRTITTGSPITYSTYGKFLADGGCSGGAYDIIICDECHST
DATSILGIGTVLDQAETAGARLVVLATATPPGSVTVSHPNIEEVALSTTGEIPFYGKAIPLEVIKGGRH
LIFCHSKKKCDELAAKLVALGINAVAYYRGLDVSVIPTSGDVVVVST DALMTGFTGDFDSV
NS3-6 (which is located in positions 1192-1478 of the HCV amino acid sequence, SEQ ID
NO: 10):
AVDFIPVETMETTMRSPVFTDNSSPPAVPQTFQVAHLHAPTGSGKSTKVPAAYAAQGYKVLVL
NPSVAATLGFGAYMSKAHGVDPNIRTGVRTITTGAPITYSTYGKFLADGGCSGGAYDIIICDECHST
DSTTILGIGTVLDQAETAGARLVVLATATPPGSVTVPHPNIEEVALSNTGEIPFYGKAIPIEAIRGGRH
LIFCHSKKKCDELAAKLSSLGLNAVAYYRGLDVSVIPSSGDVVVVATDALMTGFTGDFDSVIDCNTC
VTQTVDFSLDPTFTIETTTVP
NS3-6B (which is located in positions 1192-1478 of the HCV amino acid sequence, SEQ ID
NO: 11):
AVDFIPVENLETTMRSPVFTDNSSPPAVPQSFQVAHLHAPTGSGKSTKVPAAYAAQGYKVLVL
NPSVAATLGFGAYMSKAHGVDPNIRTGVRTITTGSPITYSTYGKFLADGGCSGGAYDIIICDECHST
DATSILGIGTVLDQAETAGARLVVLATATPPGSVTVSHPNIEEVALSTTGEIPFYGKAIPLEVIKGGRH
LIFCHSKKKCDELAAKLVALGINAVAYYRGLDVSVIPTSGDVVVVSTDALMTGFTGDFDSVIDCNTC
VTQTVDFSLDPTFTIETTTLP
NS3-7 (which is located in positions 1377-1443 of the HCV amino acid sequence, SEQ ID
NO: 12):
GKAIPLEVIKGGRHLIFCHSKKKCDELAAKLVALGINAVAYYRGLDVSVIPTSGDVVVVATDALM
TG
NS3-7B (which is located in positions 1377-1443 of the HCV amino acid sequence, SEQ ID
NO: 13):
GKAIPLELIKGGRHLIFCHSKKKCDELARQLTSLGLNAVAYYRGLDVSVIPTSGDVVVCATDAL
MTG
NS3-8 (which is located in positions 1201-1465 of the HCV amino acid sequence, SEQ ID
NO: 14):
LETTMRSPVFTDNSSPPAVPQSFQVAHLHAPTGSGKSTKVPAAYAAQGYKVLVLNPSVAATLG
FGAYMSKAHGVDPNIRTGVRTITTGSPITYSTYGKFLADGGCSGGAYDIIICDECHSTDATSILGIGT
VLDQAETAGARLVVLATATPPGSVTVSHPNIEEVALSTTGEIPFYGKAIPLEVIKGGRHLIFCHSKKKC
DELAAKLVALGINAVAYYRGLDVSVIPTSGDVVVVSTDALMTGFTGDFDSVIDCNTCVTQTVDFS
NS4-1 (which is located in positions 1698-1735 of the HCV amino acid sequence, SEQ ID
NO: 15):
REVLYREFDEMEECSQHLPYIEQGMMLAEQFKQKALGL
NS4-2 (which is located in positions 1658-1949 of the HCV amino acid sequence, SEQ ID
NO: 16):
STWVLVGGILAALAAYCLSAGCVVIVGRIILSGKPAIIPDREVLYREFDEMEECSQHLPYIEQGM
MLAEQFKQKALGLLQTASRQAEATAPVVQSKWQSLETFWAKHMWNFISGIQYLAGLSTLPGNPAI
ASLMAFTAAVTSPFSTQQTLLFNILGGWVAAQLAAPSAATSFVGAGIAGAAIGSVGLGKVLVDILAG
YGAGVAGALVAFKVMSGEVPTTEDLVNLLPAILSPGALVVGWVCAAILRRHVGPGEGAVQWMNRLI
AFASRGNHVSPTHYVPESDAAARVTTILS
NS4-3 (which is located in positions 1691-1749 of the HCV amino acid sequence, SEQ ID
NO: 17):
KPAIIPDREVLYREFDEMEECSQHLPYIEQGMMLAEQFKQKALGLLQTATKQAEAAAPV
NS4-3B (which is located in positions 1691-1749 of the HCV amino acid sequence, SEQ ID
NO: 18):
KPAVIPDREVLYREFDEMEECSSHLPYIEQGMQLAEQFKQKALGLLQTATKQAEAAAPV
NS4-4 (which is located in positions 1695-1741 of the HCV amino acid sequence, SEQ ID
NO: 19):
GGKPALVPDKEVLYQQYDEMEECSQAAPYIEQAQVIAHQFKEKVLGL
NS4-5 (which is located in positions 1693-1740 of the HCV amino acid sequence, SEQ ID
NO: 20):
NDQVWTPDKEILYEAFDEMEECASKAALIEEGQRMAEMLKSKIQGLL
NS4-6 (which is located in positions 1698-1931 of the HCV amino acid sequence, SEQ ID
NO: 21):
REVLYREFDEMEECSQHLPYIEQGMMLAEQFKQKALGLLQTASRQAEATAPVVQSKWQSLE
TFWAKHMWNFISGIQYLAGLSTLPGNPAIASLMAFTAAVTSPFSTQQTLLFNILGGWVAAQLAAPSA
ATSFVGAGIAGAAIGSVGLGKVLVDILAGYGAGVAGALVAFKVMSGEVPTTEDLVNLLPAILSPGALV
VGWVCAAILRRHVGPGEGAVQWMNRLIAFASRGNHVSP
NS4-7 (which is located in positions 1691-1799 of the HCV amino acid sequence, SEQ ID
NO: 22):
KPAIIPDREVLYREFDEMEECSQHLPYIEQGMMLAEQFKQKALGLLQTASRQAEATAPVVQSK
WQSLETFWAKHMWNFISGIQYLAGLSTLPGNPAIASLMAFTAAVTS
NS4-8 (which is located in positions 1929-1935 of the HCV amino acid sequence, SEQ ID
NO: 23):
VSPTHYV
Core-1 (which is located in positions 8-34 of the HCV amino acid sequence, SEQ ID NO: 24):
QRKTKRNTDRRPQDVKFPGGGQIVGGV
Core-2 (which is located in positions 1-179 of the HCV amino acid sequence, SEQ ID NO: 25):
MSTNPKPQRKTKRNTDRRPQDVKFPGGGQIVGGVYLLPRRGPRLGVRATRKTSERSQPRG
RRQPIPKARRPEGRTWAQPGYPWPLYGNEGFGWAGWLLSPRGSRPSWGPTDPRRRSRNLGKV
IDTLTCGFADLMGYIPLVGAPLGGAARALAHGVRVLEDGVNYATGNLPGCSFSIFLL
Core-2B (which is located in positions 1-179 of the HCV amino acid sequence, SEQ ID NO: 26):
MSTNPKPQRKTKRNTNRRPQDVKFPGGGQIVGGVYLLPRRGPRLGVRATRKTSERSQPRG
RRQPIPKARRPEGRTWAQPGYPWPLYGNEGMGWAGWLLSPRGSRPSWGPTDPRRRSRNLGKV
IDTLTCGFADLMGYIPLVGAPLGGAARALAHGVRVLEDGVNYATGNLPGCSFSIFLL
Core-3 (which is located in positions 1-130 of the HCV amino acid sequence, SEQ ID NO: 27):
MSTNPKPQRKTKRNTDRRPQDVKFPGGGQIVGGVYLLPRRGPRLGVRATRKTSERSQPRG
RRQPIPKARRPEGRTWAQPGYPWPLYGNEGFGWAGWLLSPRGSRPSWGPTDPRRRSRNLGKV
IDTLTCGF
Core-3B (which is located in positions 1-130 of the HCV amino acid sequence, SEQ ID NO: 28):
MSTNPKPQRKTKRNTNRRPQDVKFPGGGQIVGGVYLLPRRGPRLGVRATRKTSERSQPRG
RRQPIPKARRPEGRTWAQPGYPWPLYGNEGLGWAGWLLSPRGSRPSWGPTDPRRRSRNLGKVI
DTLTCGF
Core-4 (which is located in positions 1-80 of the HCV amino acid sequence, SEQ ID NO: 29):
MSTNPKPQRKTKRNTDRRPQDVKFPGGGQIVGGVYLLPRRGPRLGVRATRKTSERSQPRG
RRQPIPKARRPEGRTWAQPG
Core-4B (which is located in positions 1-80 of the HCV amino acid sequence, SEQ ID NO: 30):
MSTNPKPQRKTKRNTNRRPQDVKFPGGGQIVGGVYLLPRRGPRLGVRATRKTSERSQPRG
RRQPIPKARRPEGRTWAQPG
Core-5 (which is located in positions 1-53 of the HCV amino acid sequence, SEQ ID NO: 31):
MSTNPKPQRKTKRNTDRRPQDVKFPGGGQIVGGVYLLPRRGPRLGVRATRKTS
Core-6 (which is located in positions 1-48 of the HCV amino acid sequence, SEQ ID NO: 32):
MSTNPKPQRKTKRNTDRRPQDVKFPGGGQIVGGVYLLPRRGPRLGVRA
Core-7 (which is located in positions 8-60 of the HCV amino acid sequence, SEQ ID NO: 33):
QRKTKRNTNRRPQDVKFPGGGQIVGGVYLLPRRGPRLGVRATRKTSERSQPRG
Core-8 (which is located in positions 1-34 of the HCV amino acid sequence, SEQ ID NO: 34):
MSTNPKPQRKTKRNTDRRPQDVKFPGGGQIVGGV

### Embodiment 3 HCV Recombinant Protein Induction Expression and Purification

Recombinant protein induction expression: the constructed expression plasmids are transformed into E. coli BL21 competent cells (New England Biolabs (NEB), article number: C2530H) by means of a thermal stimulation method, coated on an LB plate containing 50 ug/ml Kan, and is cultured for 16 h at 37° C. Single colonies are selected, positive strains identified by PCR and double digestion of a bacterial solution are selected for conservation and inoculated in an LB culture medium containing 50 ug/ml Kan, and shaking culture is performed at 37° C. After 0D600 reaches 0.6-0.8, 1.0 mM IPTG is added, and culture is performed for 2-4h at 37° C., so as to induce protein expression; and the total protein is extracted, and the expression of the recombinant protein is identified by means of SDS-PAGE. By means of a 6*HIS tag on the N terminal of the recombinant protein, the protein with the purity being 96% is obtained after nickel ion chelation purification and SP column purification.

### Embodiment 4 Evaluation of the Sensitivity of a Double Antigen Sandwich Assay for HCV Recombinant Protein for Detection of Hepatitis C Antibodies

The purified HCV recombinant antigen is prepared into an HCV colloidal gold lateral chromatography test strip. A specific process includes the following:

4.1 Colloidal Gold Preparation

100 ml of ultrapure water is added to a conical flask, and then is heated to boiling on a magnetic heating stirrer; 1 ml of 1% chloroauric acid (Sigma-Aldrich, article number: 16961-25-4) solution is added, 1 ml of 1% sodium citrate (Sigma-Aldrich, article number: 6132-04-3) aqueous solution is immediately added after boiling; continuous boiling is kept for 10 minutes; and then natural cooling is performed.

4.2 Preparation of an HCV Recombinant Antigen-Colloidal Gold Conjugate

10 ml of the colloidal gold is taken and placed into a beaker, 170 μl of 0.2M K₂CO₃ is added during stirring, so as to adjust pH to 7.5, and stirring is continuously performed for 20 seconds; a certain quantity of anti-fusion protein monoclonal antibodies is added, and stirring is continuously performed for 10 minutes; 0.1 ml of 10% BSA is added, and stirring is continuously performed for 5 minutes; centrifugation is performed on 8000 g of the mixture for 20 minutes, supernatant is discarded, and the precipitation is made up to 1 ml bycolloidal gold diluent (20 mM PB, 150 mM NaCl, 1% BSA, 0.2% TritonX-100, 2% Sucrose, 0.01% Proclin300); and finally, a certain quantity of HCV recombinant antigen with the fusion protein is added to 1 ml of the anti-fusion protein monoclonal antibody complex marked with the colloidal gold, and is stored at −4° C. after well mixing, and the HCV recombinant antigen-colloidal gold conjugate is prepared.

4.3 Preparation of a Gold-Marked Pad

The HCV recombinant antigen-colloidal gold conjugate is diluted 10 folds with the colloidal gold diluent, then glass fiber (Watman) is immersed, freeze-drying is performed, and the gold-marked pad is prepared.

4.4 Nitrocellulose Membrane (NC Membrane) Encapsulation

A corresponding HCV recombinant antigen (which is the same as the recombinant antigen used for detection of the same group) is used as an HCV encapsulated antigen; the HCV encapsulated antigen is diluted to 0.5 mg/ml with a detection line diluent (10 mM PBS+2% sucrose), so as to prepare into a detection line working solution; and the corresponding position of an NC membrane (Millipore, article number: HF135002) is scribed by using a membrane dotting instrument, and drying is performed for 1 hour at 3710.

4.5 Assembly

The gold-marked pads are respectively assembled with the encapsulated NC membranes, absorbent papers, polyester plates and sample pads, so as to form HCV gold-marked detection reagent strips.

4.6 Detection Method

80 μl of a sample to be detected (for example, serum) is added to the sample pad, and placed for 15 minutes at room temperature, and then a result is determined.

4.7 Performance Evaluation of a Test Strip

An RIBA HCV3.0SIA reagent (CHIRON) is used, and sensitivity detection is performed by using 100 clinically positive samples with confirmed HCV.

The sensitivity is obtained by detecting the 100 RIBA-confirmed positive samples by using the recombinant antigen constructed above, and is specifically calculated as follows:

Sensitivity=[TP/(τP+FN)]×100%

Where TP is the number of samples with positive (true positive) detection results, and FN is the number of samples with negative (that is, false negative) detection results.

With regard to this embodiment, the sensitivity is the number of samples with positive detection results divided by 100 and multiplied by 100%.

Number of detections in 100 HCV-positive samples

| | Results of RIBA-confirmed antibody profiles | | | | | | | |
|---|---|---|---|---|---|---|---|---|
| Recombinant | Core | NS3 | Core + NS3 | NS3 + NS4 | Core + NS3 + NS4 | Other | Total | |
| antigen | 21 | 18 | 55 | 2 | 2 | 2 | 100 | Sensitivity |
| HCV-1 | 20 | 17 | 55 | 2 | 2 | 1 | 97 | 97% |
| HCV-1B | 20 | 17 | 55 | 2 | 2 | 1 | 97 | 97% |
| HCV-2 | 20 | 18 | 54 | 2 | 2 | 1 | 97 | 97% |
| HCV-2B | 20 | 18 | 54 | 2 | 2 | 1 | 97 | 97% |
| HCV-3 | 20 | 18 | 55 | 2 | 2 | 1 | 98 | 98% |
| HCV-3B | 20 | 18 | 55 | 2 | 2 | 1 | 98 | 98% |
| HCV-4 | 20 | 18 | 55 | 2 | 2 | 2 | 99 | 99% |
| HCV-4B | 20 | 18 | 55 | 2 | 2 | 2 | 99 | 99% |
| HCV-5 | 20 | 18 | 55 | 2 | 2 | 1 | 98 | 98% |
| HCV-5B | 20 | 18 | 55 | 2 | 2 | 1 | 98 | 98% |
| HCV-6 | 20 | 18 | 55 | 2 | 2 | 1 | 98 | 98% |
| HCV-6B | 20 | 18 | 55 | 2 | 2 | 1 | 98 | 98% |
| HCV-7 | 20 | 18 | 55 | 2 | 2 | 1 | 98 | 98% |
| HCV-7B | 20 | 18 | 55 | 2 | 2 | 1 | 98 | 98% |
| HCV-8 | 20 | 18 | 55 | 2 | 2 | 1 | 98 | 98% |
| HCV-8B | 20 | 18 | 55 | 2 | 2 | 1 | 98 | 98% |
| HCV-9 | 20 | 18 | 55 | 2 | 2 | 1 | 98 | 98% |
| HCV-9B | 20 | 18 | 55 | 2 | 2 | 1 | 98 | 98% |
| HCV-5C | 20 | 18 | 55 | 2 | 2 | 1 | 98 | 98% |
| HCV-4C | 20 | 18 | 55 | 2 | 2 | 2 | 99 | 99% |
| HCV-10 | 20 | 18 | 55 | 2 | 2 | 1 | 98 | 98% |
| HCV-11 | 19 | 17 | 54 | 1 | 1 | 1 | 93 | 93% |
| HCV-12 | 19 | 12 | 46 | 0 | 1 | 0 | 78 | 78% |
| HCV-13 | 19 | 17 | 54 | 1 | 2 | 1 | 94 | 94% |
| HCV-14 | 19 | 17 | 54 | 1 | 2 | 2 | 95 | 95% |
| HCV-15 | 0 | 17 | 41 | 1 | 1 | 1 | 61 | 61% |
| HCV-16 | 19 | 13 | 47 | 1 | 0 | 1 | 81 | 81% |
| HCV-17 | 19 | 12 | 48 | 2 | 2 | 1 | 84 | 84% |
| HCV-18 | 19 | 12 | 48 | 1 | 1 | 1 | 82 | 82% |
| HCV-19 | 19 | 13 | 48 | 2 | 2 | 1 | 85 | 85% |
| HCV-20 | 19 | 13 | 48 | 1 | 1 | 1 | 83 | 83% |

### Embodiment 5

The fusion is performed by adjusting a fusion order of fragments of different sections in HCV-4 according to the following table. The method refers to the above embodiments. Sensitivity test results are as follows.

Antigens

| obtained in | RIBA-confirmed antibody profiles | | | | | | | |
|---|---|---|---|---|---|---|---|---|
| different fusion | Core | NS3 | Core + NS3 | NS3 + NS4 | Core + NS3 + NS4 | Other | Total | |
| orders | 21 | 18 | 55 | 2 | 2 | 2 | 100 | Sensitivity |
| Core + NS3 + NS4 | 21 | 17 | 55 | 2 | 2 | 1 | 98 | 98% |
| NS3 + Core + NS4 | 21 | 17 | 55 | 2 | 2 | 1 | 98 | 98% |
| NS3 + NS4 + Core | 21 | 18 | 55 | 2 | 2 | 1 | 99 | 98% |
| Core + NS4 + NS3 | 21 | 18 | 55 | 1 | 2 | 1 | 98 | 98% |
| NS4 + Core + NS3 | 21 | 17 | 55 | 2 | 2 | 1 | 98 | 98% |
| NS4 + NS3 + Core | 21 | 17 | 55 | 2 | 2 | 1 | 98 | 98% |

### Embodiment 6

In order to further improve the specificity of antibody detection, site-directed mutation is performed for the recombinant antigen NS3 region. Using HCV-4 and HCV-7 recombinant antigens as an example, at least one Cysteine (C) in position 1305/1315/1318/1394/1400/1454 involved in two NS3 regions (NS3-5 and NS3-4) in HCV-4 or two NS3 regions (NS3-2 and NS3-6) in HCV-7 is mutated into any one amino acid of Glycine (G)/Alanine (A)/Serine (S), and the rest of the section design remains unchanged, so as to respectively obtain recombinant antigens of HCV41-HCV48 and recombinant antigens of HCV71-HCV75. The above HCV recombinant antigen is prepared as a colloidal gold test strip with reference to the above embodiment methods, and 1000 clinically negative samples are detected to obtain a specificity detection result. 100 positive samples are detected, so as to obtain a sensitivity result, and a total detection rate is calculated. The specificity shown as a percentage herein is obtained by testing 1000 known negative samples using the above HCV colloidal goldtest strips, and is calculated as follows:

Specificity=[TN/(TN+FP)]×100%

Where TN is the number of samples with negative detection results, and FP is the number of samples with positive (that is, false positive) detection results.

With regard to this embodiment, the specificity is the number of samples with negative detection results divided by 1000 and multiplied by 100%.

The recombinant antigens are prepared as described in Embodiment 2, and mutations in each recombinant antigen are described in detail below.

With regard to HCV-4, the following mutated recombinant antigens HCV41-HCV48 are obtained by means of preparation:

HCV-41 recombinant antigen, in which the mutations performed are: NS3-5 and C1318S; and NS3-4 and C1400G.

HCV-42 recombinant antigen, in which the mutations performed are: NS3-5 and C1400A; and NS3-4 and C1394S.

HCV-43 recombinant antigen, in which the mutations performed are: NS3-5 and C1315G; and NS3-4 and C1394A.

HCV-44 recombinant antigen, in which the mutations performed are: NS3-5, C1305A and C1318A; and NS3-4, C1394S and C1400S.

HCV-45 recombinant antigen, in which the mutations performed are: NS3-5, C1315G and C1318G; and NS3-4, C1394A and C1400G.

HCV-46 recombinant antigen, in which the mutations performed are: NS3-5, C1315G, C1318A, C1394S and C1400A; and NS3-4, C1394G and C1400A.

HCV-47 recombinant antigen, in which the mutations performed are: NS3-5, C1305A, C1315A, C1318A, C1394A and C1400A; and NS3-4, C1394A and C1400A.

HCV-48 recombinant antigen, in which the mutations performed are: NS3-5, C1305A, C1315G, C1318S, C1394S and C1400A; and NS3-4, C1394S and C1400A.

With regard to HCV-7, the following mutated recombinant antigens HCV71-HCV75 are obtained by means of preparation:

HCV-71 recombinant antigen, in which the mutations performed are: NS3-2 and C1454A; and NS3-6, C1305A, C1315G, C1318G, C1394S and C1400A.

HCV-72 recombinant antigen, in which the mutations performed are: NS3-2, C1305G, C1315S and C1454A; and NS3-6, C1318G, C1394S and C1400A.

HCV-73 recombinant antigen, in which the mutations performed are: NS3-2, C1318G and C1400G; and NS3-6, C1394A and C1400G.

HCV-74 recombinant antigen, in which the mutations performed are: NS3-2, C1305A, C1315A, C1318A, C1394A, C1400A and C1454A; and NS3-6, C1305A, C1315A, C1318A, C1394A, C1400A and C1454A.

HCV-75 recombinant antigen, in which the mutations performed are: NS3-2, C1305S, C1315G and C1318G; and NS3-6, C1305S, C1315A, C1318A, C1394G and C1400G.

The expression of mutations herein may be understood in light of, for example, C1305A being a mutation of C to A at position 1305 on the base of the foregoing antigen sequence.

Detection results are shown in the following table.

| Fusion antigen name | Specificity | Sensitivity | Detection rate |
|---|---|---|---|
| HCV-4 | 98.00% | 99% | 98.09% |
| HCV-41 | 98.40% | 99% | 98.45% |
| HCV-42 | 98.50% | 99% | 98.55% |
| HCV-43 | 98.80% | 99% | 98.82% |
| HCV-44 | 99.10% | 99% | 99.09% |
| HCV-45 | 99.30% | 99% | 99.27% |
| HCV-46 | 99.50% | 99% | 99.45% |
| HCV-47 | 99.80% | 99% | 99.73% |
| HCV-48 | 99.40% | 99% | 99.36% |
| HCV-7 | 98.20% | 98% | 98.18% |
| HCV-71 | 99.10% | 98% | 99.00% |
| HCV-72 | 99.00% | 98% | 98.91% |
| HCV-73 | 99.10% | 98% | 99.00% |
| HCV-74 | 99.90% | 98% | 99.73% |
| HCV-75 | 99.40% | 98% | 99.27% |

The above are only the preferred implementations of the present disclosure and are not intended to limit the disclosure. For those skilled in the art, the present disclosure may have various modifications and variations. Any modifications, equivalent replacements, improvements and the like made within the spirit and principle of the disclosure shall fall within the scope of protection of the disclosure.

### INDUSTRIAL APPLICABILITY

The method and/or kit described here may be used for detecting the presence of the HCV antibody in the sample of the subject, measuring the volume or concentration of the HCV antibody, monitoring progression of diseases, monitoring a treatment effect, and/or determining the onset of hepatitis in subjects or onset risks. By means of the method and/or products of the present disclosure, one or more problems of low sensitivity and low specificity can be solved, thereby improving sensitivity and/or specificity.

What is claimed is:

1. A Hepatitis C Virus (HCV) recombinant antigen, comprising at least two NS3 antigens, at least one NS4 antigen, and at least one core antigen.

2. The HCV recombinant antigen according to claim 1, wherein the sensitivity of the HCV recombinant antigen for detecting an HCV antibody is not less than 94.0%.

3. The HCV recombinant antigen according to claim 1, wherein
the NS3 antigen comprises polypeptide selected from positions 1075-1657 of an HCV amino acid sequence, for example, the length of the polypeptide is 41-583 amino acids, for example, 50, 60, 70, 80, 90, 100, 150, 200, 250, 300, 350, 400, 450, 500 or 550 amino acids; for example, the NS3 antigen comprises polypeptide of which initiation site is at positions 1075-1380 of the HCV amino acid sequence (for example, the initiation site is at position 1075, 1192, 1201, 1230, 1377, 1380 or any position of the positions 1075-1380) and a termination site at positions 1420-1657 of the HCV amino acid sequence (for example, the termination site is at position 1420, 1443, 1451, 1465, 1478, 1657 or any position of the positions 1420-1657); for example, the NS3 antigen comprises positions 1380-1420, 1075-1657, 1230-1465, 1377-1443, 1192-1451, 1192-1478, 1377-1443, 1201-1465, 1192-1608 or 1192-1517 of the HCV amino acid sequence; for example, the NS3 antigen comprises a sequence selected from SEQ ID NO:1-14; for example, the NS3 antigen comprises a sequence selected from sequences having at least 95%, at least 96%, at least 97%, at least 98%, or at least 99% identity to SEQ ID NO:1-14;
the NS4 antigen comprises polypeptide selected from positions 1658-1949 of an HCV amino acid sequence, for example, the length of the polypeptide is 38-292 amino acids, for example, 40, 50, 60, 70, 80, 90, 100, 150, 200 or 250 amino acids; for example, the NS4 antigen comprises polypeptide of which initiation site is at positions 1658-1698 of the HCV amino acid sequence (for example, the initiation site is at position 1658, 1691, 1693, 1695, 1698 or any position of the positions 1658-1698) and a termination site at positions 1735-1949 of the HCV amino acid sequence (for example, the termination site is at position 1735, 1740, 1741, 1749, 1799, 1931, 1935, 1949 or any position of the positions 1735-1949); for example, the NS4 antigen comprises positions 1698-1735, 1658-1949, 1691-1749, 1695-1741, 1693-1740, 1698-1931, 1691-1799 or 1929-1935 of the HCV amino acid sequence; for example, the NS4 antigen comprises a sequence selected from SEQ ID NO:15-22; for example, the NS4 antigen comprises a sequence selected from sequences having at least 95%, at least 96%, at least 97%, at least 98%, or at least 99% identity to SEQ ID NO:15-22; and/or
the core antigen comprises polypeptide selected from positions 1-179 of an HCV amino acid sequence, for example, the length of the polypeptide is 27-179 amino acids, for example, 30, 40, 50, 60, 70, 80, 90, 100 or 150 amino acids; for example, the core antigen comprises polypeptide of which initiation site is at positions 1-8 of the HCV amino acid sequence (for example, the initiation site is at position 1, 8 or any position of the positions 1-8) and a termination site at positions 34-179 of the HCV amino acid sequence (for example, the termination site is at position 34, 48, 53, 60, 80, 130, 179 or any position of the positions 34-179); for example, the core antigen comprises positions 1-34, 8-34, 1-179, 1-130, 1-80, 1-53, 1-48 or 8-60 of the HCV amino acid sequence; for example, the core antigen comprises a sequence selected from SEQ ID NO:24-34; for example, the core antigen comprises a sequence selected from sequences having at least 95%, at least 96%, at least 97%, at least 98%, or at least 99% identity to SEQ ID NO:24-34.

4. The HCV recombinant antigen according to claim 1, wherein at least any one of cysteine in the NS3 antigen is mutated into another amino acid, for example, mutated into G, A or S; for example, at least any one of cysteine of the NS3 antigen in position 1305/1315/1318/1394/1400/1454 of the corresponding HCV amino acid sequence is mutated into another amino acid, for example, mutated into G, A or S.

5. The HCV recombinant antigen according to claim 4, wherein the specificity of the HCV recombinant antigen for detecting the HCV antibody is not less than 98.0%.

6. The HCV recombinant antigen according to claim 4, wherein the sensitivity of the HCV recombinant antigen for detecting the HCV antibody is not less than 98.0%, for example, not less than 99.0%.

7. The HCV recombinant antigen according to claim 1, wherein the NS3 antigen, the NS4 antigen and the core antigen are directly connected or connected optionally by means of one or more linkers, for example, the linker is (G)n, (GS)n, (SG)n, (GGGS)n or (GGGGS)n, wherein n is 1, 2, 3, 4, 5, 6, 7, 8, 9, 10 or a larger integer.

8. The HCV recombinant antigen according to one of claim 1, comprising N- and/or C-terminal modifications, for example, histidine tags, biotinylation and/or detectable marker modifications.

9. The HCV recombinant antigen according to claim 1, wherein HCV comprises an HCV genotype 1a, 1 b, 2a, 2b, 2c, 2d, 3a, 3b, 3c, 3d, 3e, 3f, 4a, 4b, 4c, 4d, 4e, 4f, 4g, 4h, 4i, 4j, 5a or 6a.

10. The HCV recombinant antigen according to claim 1, wherein contained antigens are fused in the order of the NS3 antigen, the NS4 antigen, and the core antigen from an N-terminal to a C-terminal.

11. A nucleic acid encoding the Hepatitis C Virus (HCV) recombinant antigen according to claim 1, or an expression vector containing the nucleic acid, or a host cell containing the expression vector.

12. A conjugate, comprising the Hepatitis C Virus (HCV) recombinant antigen according to claim 1.

13. The conjugate according to claim 12, comprising a solid support, a detectable marker or a binding partner which is conjugated with the HCV recombinant antigen; for example, the HCV recombinant antigen in the conjugate is directly or indirectly conjugated; for example, the solid support comprises magnetic particles, a microtiter plate or a cellulose membrane; for example, the detectable marker is a metal particle, a fluorescent marker, a chromophore marker, an electron-dense marker, a chemiluminescent marker, a radioactive marker, or an enzyme marker, for example, the detectable marker is colloidal gold, radio isotope, fluorophores, a spin marker, or a bacteriophage marker, for example, the detectable marker is rhodamine, fluorescein, acridinium ester, luciferase, horseradish peroxidase, alkaline phosphatase, β-galactosidase, glucoamylase, lysozyme, carbohydrate oxidase, glucose oxidase, galactose oxidase or a glucose-6-phosphate dehydrogenase marker; and for example, the binding partner comprises biotin, streptavidin or avidin.

14. A kit, comprising the Hepatitis C Virus (HCV) recombinant antigen according to claim 1, or a conjugate comprising the Hepatitis C Virus (HCV) recombinant antigen according to claim 1.

15. (canceled)

16. (canceled)

17. A method for detecting a Hepatitis C Virus (HCV) antibody or antigen in a sample of a subject, comprising contacting the HCV recombinant antigen according to claim 1 or a conjugate comprising the Hepatitis C Virus (HCV) recombinant antigen according to claim 1.

18. A method for diagnosing hepatitis C, comprising:
using the Hepatitis C Virus (HCV) recombinant antigen according to claim 1 or the conjugate comprising the Hepatitis C Virus (HCV) recombinant antigen according to claim 1 to detect an HCV antibody or antigen from a sample of a subject; and
analyzing a detection result.

19. The HCV recombinant antigen according to claim 1, wherein the HCV recombinant antigen comprises at least two NS3 antigens, at least two NS4 antigens, and at least one core antigen; optionally, the HCV recombinant antigen comprises at least two NS3 antigens, at least three NS4 antigen, and at least one core antigen; optionally, the number of the NS3 antigens in the HCV recombinant antigen is two, three or more, the number of the NS4 antigens in the HCV recombinant antigen is two, three or more.

20. The HCV recombinant antigen according to claim 1, wherein the two, three or more NS3 antigens are the same or different, preferably different; preferably, the two, three or more NS4 antigens are different.

21. The HCV recombinant antigen according to claim 1, wherein the sensitivity of the HCV recombinant antigen for detecting an HCV antibody is not less than 97.0%.

22. The HCV recombinant antigen according to claim 1, wherein the sensitivity of the HCV recombinant antigen for detecting an HCV antibody is not less than 98.0%, or not less than 99.0%.


SEQUENCE LISTING (36 sequences)

SEQ ID NO: 1 - protein, 41 aa, Hepatitis C virus
IPIEAIRGGRHLIFCHSKKKCDELAAKLSSLGLNAVAYYRG

SEQ ID NO: 2 - protein, 583 aa, Hepatitis C virus
NGVCWTVYHGAGSKTLAGPKGPITQMYTNVDQDLVGWHRPPGARSLTPCTCGSSDLYLVTRHADVIPVRRRGDSRGSLLSPRPVSYLKGSSGGPLLCPFGHVAGIFRAAVCTRGVAKAVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTCVTQTVDFSLDPTFTIETTTVPQDAVSRSQRRGRTGRGREGIYRFVTPGERPSGMFDSSVLCECYDAGCAWYELTPAETTVRLRAYLNTPGLPVCQDHLEFWEGVFTGLTHIDAHFLSQTKQAGDNFPYLVAYQATVCAKAQAPPPSWDQMWKCLTRLKPTLQGPTPLLYRLGAVQNEVTLTHPITKYIMTCMSADLEVVT

SEQ ID NO: 3 - protein, 583 aa, Hepatitis C virus
NGVCWTVYHGAGSKTLAGPKGPITQMYTNVDQDLVGWQAPPGARSLTPCTCGSSDLYLVTRHADVIPVRRRGDTRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGGPITYSTYGKFLADGGCSGGAYDIIICDECHSTDSTTILGIGTVLDQAETAGARLVVLATATPPGSVTVPHPNIEEVALSNTGEIPFYGKAIPIETIKGGRHLIFCHSKKKCDELAAKLSSLGLNAVAYYRGLDVSVIPTSGDVVVVATDALMTGFTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLTRLKPTLHGPTPLLYRLGAVQNEVTLTHPITKYIMACMSADLEVVT

SEQ ID NO: 4 - protein, 236 aa, Hepatitis C virus
APTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTCVTQTVDFS

SEQ ID NO: 5 - protein, 236 aa, Hepatitis C virus
APTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFS

SEQ ID NO: 6 - protein, 67 aa, Hepatitis C virus
GKAIPIEAIRGGRHLIFCHSKKKCDELAAKLSSLGLNAVAYYRGLDVSVIPSSGDVVVVATDALMTG

SEQ ID NO: 7 - protein, 67 aa, Hepatitis C virus
GKAIPLEVIKGGRHLIFCHSKKKCDELAAKLVALGINAVAYYRGLDVSVIPTSGDVVVVSTDALMTG

SEQ ID NO: 8 - protein, 260 aa, Hepatitis C virus
AVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSV

SEQ ID NO: 9 - protein, 260 aa, Hepatitis C virus
AVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSV

SEQ ID NO: 10 - protein, 287 aa, Hepatitis C virus
AVDFIPVETMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEAIRGGRHLIFCHSKKKCDELAAKLSSLGLNAVAYYRGLDVSVIPSSGDVVVVATDALMTGFTGDFDSVIDCNTCVTQTVDFSLDPTFTIETTTVP

SEQ ID NO: 11 - protein, 287 aa, Hepatitis C virus
AVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLP

SEQ ID NO: 12 - protein, 67 aa, Hepatitis C virus
GKAIPLEVIKGGRHLIFCHSKKKCDELAAKLVALGINAVAYYRGLDVSVIPTSGDVVVVATDALMTG

SEQ ID NO: 13 - protein, 67 aa, Hepatitis C virus
GKAIPLELIKGGRHLIFCHSKKKCDELARQLTSLGLNAVAYYRGLDVSVIPTSGDVVVCATDALMTG

SEQ ID NO: 14 - protein, 265 aa, Hepatitis C virus
LETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFS

SEQ ID NO: 15 - protein, 38 aa, Hepatitis C virus
REVLYREFDEMEECSQHLPYIEQGMMLAEQFKQKALGL

SEQ ID NO: 16 - protein, 292 aa, Hepatitis C virus
STWVLVGGILAALAAYCLSAGCVVIVGRIILSGKPAIIPDREVLYREFDEMEECSQHLPYIEQGMMLAEQFKQKALGLLQTASRQAEATAPVVQSKWQSLETFWAKHMWNFISGIQYLAGLSTLPGNPAIASLMAFTAAVTSPFSTQQTLLFNILGGWVAAQLAAPSAATSFVGAGIAGAAIGSVGLGKVLVDILAGYGAGVAGALVAFKVMSGEVPTTEDLVNLLPAILSPGALVVGVVCAAILRRHVGPGEGAVQWMNRLIAFASRGNHVSPTHYVPESDAAARVTTILS

SEQ ID NO: 17 - protein, 59 aa, Hepatitis C virus
KPAIIPDREVLYREFDEMEECSQHLPYIEQGMMLAEQFKQKALGLLQTATKQAEAAAPV

SEQ ID NO: 18 - protein, 59 aa, Hepatitis C virus
KPAVIPDREVLYREFDEMEECSSHLPYIEQGMQLAEQFKQKALGLLQTATKQAEAAAPV

SEQ ID NO: 19 - protein, 47 aa, Hepatitis C virus
GGKPALVPDKEVLYQQYDEMEECSQAAPYIEQAQVIAHQFKEKVLGL

SEQ ID NO: 20 - protein, 48 aa, Hepatitis C virus
NDQVVVTPDKEILYEAFDEMEECASKAALIEEGQRMAEMLKSKIQGLL

SEQ ID NO: 21 - protein, 234 aa, Hepatitis C virus
REVLYREFDEMEECSQHLPYIEQGMMLAEQFKQKALGLLQTASRQAEATAPVVQSKWQSLETFWAKHMWNFISGIQYLAGLSTLPGNPAIASLMAFTAAVTSPFSTQQTLLFNILGGWVAAQLAAPSAATSFVGAGIAGAAIGSVGLGKVLVDILAGYGAGVAGALVAFKVMSGEVPTTEDLVNLLPAILSPGALVVGVVCAAILRRHVGPGEGAVQWMNRLIAFASRGNHVSP

SEQ ID NO: 22 - protein, 109 aa, Hepatitis C virus
KPAIIPDREVLYREFDEMEECSQHLPYIEQGMMLAEQFKQKALGLLQTASRQAEATAPVVQSKWQSLETFWAKHMWNFISGIQYLAGLSTLPGNPAIASLMAFTAAVTS

SEQ ID NO: 23 - protein, 7 aa, Hepatitis C virus
VSPTHYV

SEQ ID NO: 24 - protein, 27 aa, Hepatitis C virus
QRKTKRNTDRRPQDVKFPGGGQIVGGV

SEQ ID NO: 25 - protein, 179 aa, Hepatitis C virus
MSTNPKPQRKTKRNTDRRPQDVKFPGGGQIVGGVYLLPRRGPRLGVRATRKTSERSQPRGRRQPIPKARRPEGRTWAQPGYPWPLYGNEGFGWAGWLLSPRGSRPSWGPTDPRRRSRNLGKVIDTLTCGFADLMGYIPLVGAPLGGAARALAHGVRVLEDGVNYATGNLPGCSFSIFLL

SEQ ID NO: 26 - protein, 179 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLL

SEQ ID NO: 27 - protein, 130 aa, Hepatitis C virus
MSTNPKPQRKTKRNTDRRPQDVKFPGGGQIVGGVYLLPRRGPRLGVRATRKTSERSQPRGRRQPIPKARRPEGRTWAQPGYPWPLYGNEGFGWAGWLLSPRGSRPSWGPTDPRRRSRNLGKVIDTLTCGF

SEQ ID NO: 28 - protein, 130 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPTDPRRRSRNLGKVIDTLTCGF

SEQ ID NO: 29 - protein, 80 aa, Hepatitis C virus
MSTNPKPQRKTKRNTDRRPQDVKFPGGGQIVGGVYLLPRRGPRLGVRATRKTSERSQPRGRRQPIPKARRPEGRTWAQPG

SEQ ID NO: 30 - protein, 80 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPG

SEQ ID NO: 31 - protein, 53 aa, Hepatitis C virus
MSTNPKPQRKTKRNTDRRPQDVKFPGGGQIVGGVYLLPRRGPRLGVRATRKTS

SEQ ID NO: 32 - protein, 48 aa, Hepatitis C virus
MSTNPKPQRKTKRNTDRRPQDVKFPGGGQIVGGVYLLPRRGPRLGVRA

SEQ ID NO: 33 - protein, 53 aa, Hepatitis C virus
QRKTKRNTNRRPQDVKFPGGGQIVGGVYLLPRRGPRLGVRATRKTSERSQPRG

SEQ ID NO: 34 - protein, 34 aa, Hepatitis C virus
MSTNPKPQRKTKRNTDRRPQDVKFPGGGQIVGGV

SEQ ID NO: 35 - protein, 15 aa, Artificial Sequence
  Synthesized
GGGGSGGGGSGGGGS

SEQ ID NO: 36 - protein, 109 aa, Artificial Sequence
  Synthesized
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA